(19) United States
(12) Patent Application Publication
(10) Pub. No.: US 2015/0297447 A1
(43) Pub. Date: Oct. 22, 2015

(54) SPACE SAVING SAUNA WITH STORAGE

(71) Applicant: Roy E. Corso, Lake Worth, FL (US)

(72) Inventor: Roy E. Corso, Lake Worth, FL (US)

(21) Appl. No.: 14/688,389

(22) Filed: Apr. 16, 2015

Related U.S. Application Data
(60) Provisional application No. 61/981,257, filed on Apr. 18, 2014.

(51) Int. Cl. A61H 33/06; A61N 5/06

(52) U.S. Cl. CPC A61H 33/066; A61N 5/0616; A61H 2201/0228; A61H 2201/0207; A61H 2201/0138; A61H 2201/0142; A61N 2005/0652

(57) ABSTRACT

A space saving sauna unit for delivering infrared light therapy to the body of a user is integrated with a piece of furniture and is sized for single occupancy, wherein a single user may receive infrared light therapy via a plurality of infrared heat sources housed in the enclosed portion of the sauna unit. The sauna enclosure may be integrated with any suitable piece of furniture, such as, but not limited to, a dresser, desk, television armoire, bed, or other piece of furniture. The plurality of infrared heat sources are operated by a set of controls and may include a time-lapsed automatic shut down feature for automatically deactivating the infrared heat sources after a predetermined amount of time has lapsed. A manually operated vent system allows for temperature control. The sauna unit may also include one or more interior audio speakers and an LED light therapy mask.

### BACKGROUND OF THE INVENTION

1. Field of the Invention

The present invention relates to sauna therapy and, more particularly, a standalone space saving sauna unit that provides for storage and which may also function as an article of furniture.

2. Discussion of the Related Art

The benefits of sauna therapy are commonly accepted amongst health practitioners. Saunas are generally used for detoxifying and eliminating impurities, such as pesticides and other chemicals, as well as heavy metals. Other benefits of sauna therapy include increased oxygenation, enhancement of the immune system, pain relief, stress reduction and weight loss. Ongoing research of the benefits of using infrared light therapy in saunas is suggestive of additional benefits. While frequent use of infrared light sauna therapy is beneficial, an in-home sauna, even a single occupant sauna, can require a considerable amount of extra floor space which might otherwise be used for a dresser, desk, television armoire, bed, or other piece of furniture.

Accordingly, there is a need for a single occupant infrared sauna that is integrated with a piece of furniture, such as a dresser, desk, television armoire or bed and which makes efficient use of space within a portion of an existing room, such as a bedroom, in a house, condominium, apartment, yacht, medical office, health or beauty spa.

### Objects and Advantages of the Invention

Considering the foregoing, it is a primary object of the present invention to provide a space saving, standalone sauna unit or custom built-in unit (e.g., within a wall).

It is a further object of the present invention to provide a space saving sauna unit that is integrated with an article of furniture to provide the dual function of a sauna and the particular purpose of the furniture article (e.g., a desk, dresser, armoire, bed).

It is a further object of the present invention to provide a space saving sauna unit that includes storage space.

It is still a further object of the present invention to provide a space saving sauna unit for delivering infrared light therapy to a user's body.

These and other objects and advantages of the present invention are more readily apparent with reference to the following detailed description and the accompanying drawings.

### SUMMARY OF THE INVENTION

The present invention is directed to a space saving sauna unit for delivering infrared light therapy to the body of a user. In several embodiments, the space saving sauna unit is integrated with a piece of furniture and is sized for single occupancy, wherein a single user may receive infrared light therapy via a plurality of infrared heat sources housed in the enclosed portion of the sauna unit. The sauna unit may be integrated with any suitable piece of furniture, such as, but not limited to, a dresser, desk, television armoire, bed, or other piece of furniture. Alternatively, the space saving sauna made be constructed as a custom built-in unit, such as within a wall in a room or hallway. The plurality of infrared lights are operated by a set of controls and may include a time-lapsed automatic shut down feature for automatically deactivating the infrared heat sources after a predetermined amount of time has lapsed. A manually operated ventilation assembly, operated by the user while in the sauna, is structured to selectively release heat from a top of the unit or contain the heat so that it is forced down into the sauna enclosure, thereby allowing the user to adjust the temperature level within the sauna. The sauna may also be equipped with one or more audio speakers (e.g., wireless speakers) and wiring with connectors for connecting a digital audio storage and playback device, such as an MP3 player or cell phone, to the audio speaker(s). Additionally, the sauna may be equipped with an LED light therapy device for anti-aging phototherapy treatment (e.g., facial phototherapy treatment).

### BRIEF DESCRIPTION OF THE DRAWINGS

For a fuller understanding of the nature of the present invention, reference should be made to the following detailed description taken in conjunction with the accompanying drawings in which:

FIG. 1 is a front elevational view of one embodiment of the space saving sauna unit of the present invention, wherein a sauna unit is integrated with a dresser furniture piece;

FIG. 2 is a side elevational view of the embodiment of the space saving sauna unit shown in FIG. 1;

FIG. 3 is a front elevational view of another embodiment of the space saving sauna unit of the present invention, wherein an empty space is provided below the sauna for storage;

FIG. 4 is a front elevational view of yet another embodiment of the space saving sauna unit, wherein the sauna is integrated with a desk having a pull-out desk surface and drawers;

FIG. 5 is a side elevational view of the space saving sauna unit of FIG. 4, shown with the pull-out desk surface extended out in an operable position;

FIG. 6 is a side elevational view of one embodiment of the space saving sauna unit of the present invention, wherein a sauna unit is integrated with a bed furniture piece;

FIG. 7 is an isolated perspective view of a portion of the interior of the sauna enclosure showing a shelf structure on the end wall with an audio speaker and a separate shelf for placement and connection of a digital audio storage and playback device such as an MP3 player or cell phone;

FIG. 8 is an isolated perspective view of the ceiling within the interior of the sauna enclosure showing an arrangement of infrared heat emitting sources, such as infrared lights, along with controls and an optional LED light therapy device;

FIG. 9 is an isolated cross sectional view showing a top ventilation chamber above the sauna enclosure and illustrating operation of air intake fans for drawing air into the ventilation chamber;

FIG. 10 is an isolated cross sectional view of the ventilation chamber and showing a vent assembly in an open position to release heat from the infrared heat generating sources out through the top of the unit;

FIG. 11 is an isolated cross sectional view of the ventilation chamber showing the vent assembly in a closed position to direct heat downwardly through the infrared heat generating sources and through the ceiling of the sauna enclosure and into the sauna interior; and

FIG. 12 is a schematic top plan view of the electrical system of space saving sauna unit;

Like reference numerals refer to like parts throughout the several views of the drawings.

### DETAILED DESCRIPTION OF THE PREFERRED EMBODIMENTS

Referring to the several views of the drawings, the space saving sauna unit with storage of the present invention is shown and is generally indicated as 10.

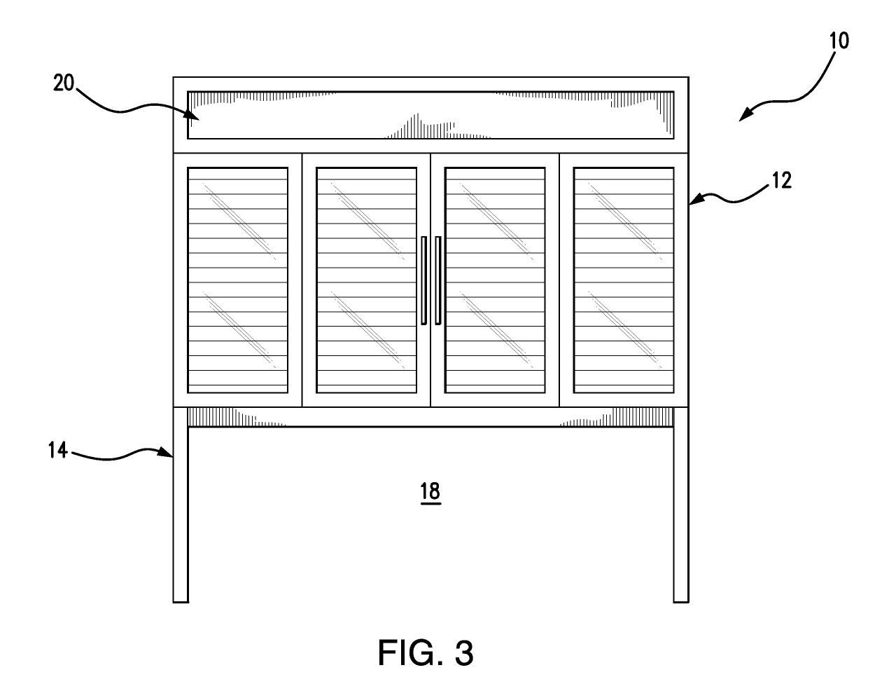

In several preferred embodiments of the invention, the space saving sauna 10 includes a sauna enclosure 12 combined with an article of furniture 14. An important aspect of the invention is the provision for storage as part of the space saving sauna unit 10. In several preferred embodiments of the invention, storage is provided directly below the sauna enclosure 12 and may be in the form of one or more drawers 16 (e.g., dresser drawers or desk drawers), one or more cabinets, or an open storage space 18 (see FIG. 3). The space saving sauna unit 10 further includes a top ventilation chamber 20 integrated with the sauna enclosure 12. The top ventilation chamber 20 is positioned directly above the sauna enclosure 12.

The sauna enclosure 12 includes a front access opening that extends substantially the entire length of the enclosure as well as substantially the entire height of the sauna enclosure. Front doors 22 are operable to an open position to allow access, including ingress and egress from the sauna enclosure. When not in use, the doors 22 can be left open or closed. During use of the sauna enclosure 12, the doors 22 are closed once the user is inside of the sauna enclosure 12 to thereby completely close the access opening. The front doors 22 are preferably mounted on bi-fold hinges so that the two left door panels fold open to the left and the two right door panels fold open to the right. Within the sauna enclosure 12, there are opposite end walls 30, 32, a back wall 34, a ceiling 36 and a flat bottom surface 38. In a preferred embodiment, the walls, ceiling and flat bottom surface are constructed of cedar wood or other natural materials that are suitable for a sauna environment.

The sauna enclosure 12 is heated to a desired temperature with the use of a plurality of infrared heat sources 40. The infrared heat sources 40 may include, but are not limited to, near infrared incandescent light bulbs, infrared ceramic tube heaters or far infrared carbon emitters that may be added to the back wall of the sauna. In the several embodiments shown in the drawings, the infrared heat sources 40 are shown as infrared light fixtures that use an infrared light emitting bulb. The infrared light fixtures are in the form of a recessed lighting fixture, commonly referred to as a hi-hat, that is mounted within the ceiling 36 of the sauna enclosure 12 so that the infrared light fixture extends up and into the top ventilation chamber 20.

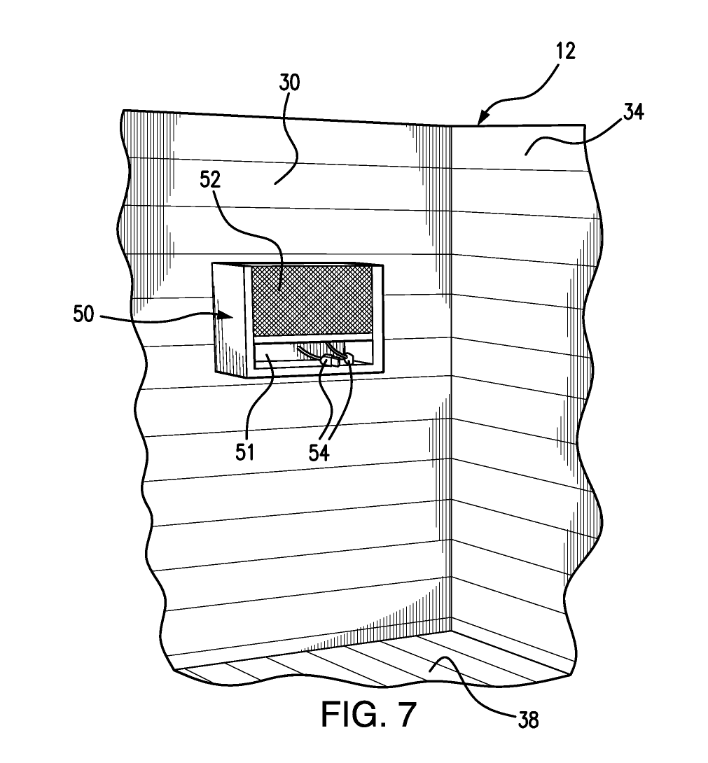

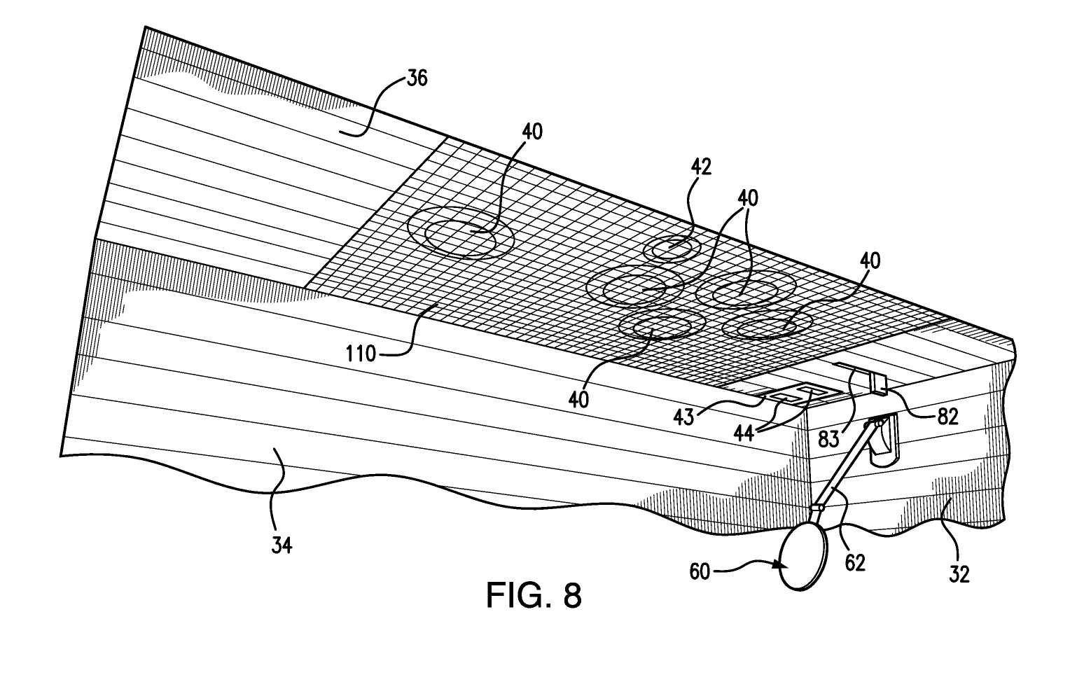

The interior of the sauna enclosure 12 may include additional components such as a wireless (e.g., Bluetooth) speaker 52 supported on a shelf structure 50 on one of the end walls 30, as seen in FIG. 7. The lower shelf 51 of the shelf structure 50 may include one or more connector cables 54 with appropriate connecting jacks for attachment to a digital audio storage and playback device, such as a MP3 player or cell phone. The digital audio playback and storage device can be placed on the lower shelf 51, below the wireless speaker 52. The opposite end wall 32 may be provided with a light therapy device 60, such as a phototherapy device for anti-aging facial skin therapy. As seen in FIG. 8, the light therapy device 60 is supported on an articulating arm member 62 that allows positioning of the light emitting panel of the light therapy device to direct the light on the user's face while the user relaxes in the sauna. The light therapy device is electronically powered and may plug into an AC outlet separated or it may be wired into the circuit of the sauna unit 10 with the lights, fans and speaker as shown in FIG. 12.

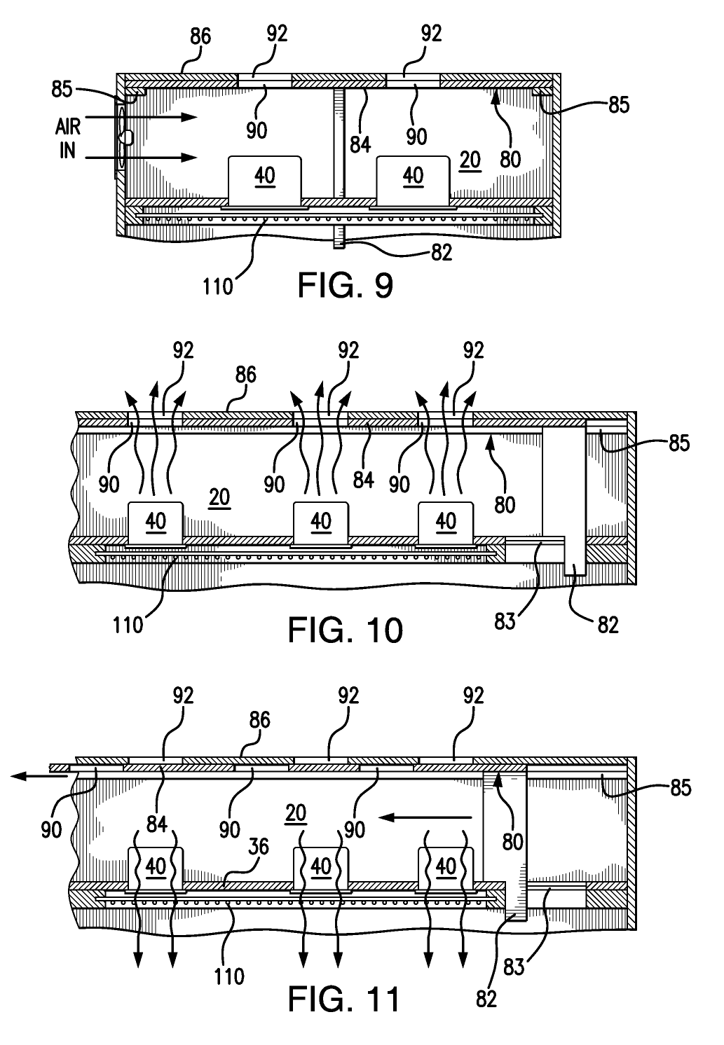

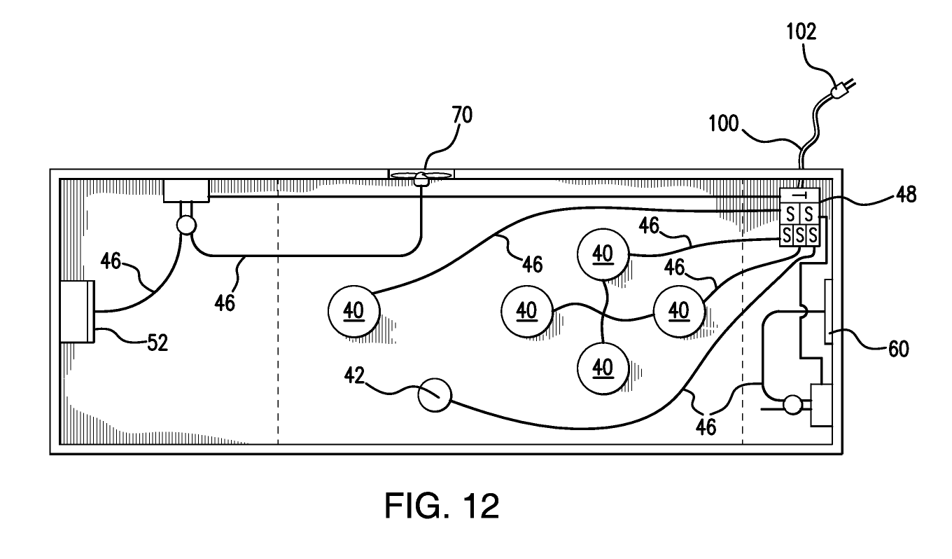

In a preferred embodiment, two or more air intake fans 70 are provided on the back wall of the top ventilation chamber 20 for drawing outside air into the air ventilation chamber, as seen in FIG. 9. Operation of the infrared heat sources 40 generates a significant amount of waste heat within the air ventilation chamber 20 which is either vented out through the top of the unit or down into the sauna enclosure 12 with the use of a vent system 80. As seen in FIGS. 8-11, the vent system 80 includes a handle 82 that extends down through a slot 83 formed in the ceiling of the sauna enclosure. The handle 82 connects to a sliding vent panel 84 fitted within tracks 85 in the top of the ventilation chamber. This vent panel 84 is slidable in relation to the top cover 86 of the sauna unit 10, as indicated by the arrows shown in FIG. 11. Both the top cover 86 of the sauna unit 10 and the sliding vent panel 84 are provided with an arrangement of openings which correspond to the position of the infrared heat sources. With the vent assembly in an open position, as seen in FIG. 10, the openings 90 in the vent panel 84 align with the openings 92 in the top cover of the sauna unit to allow escape of waste heat from the infrared heat sources 40 out through the top of the sauna unit 10. In the fully closed position, as seen in FIG. 11, the vent panel 84 has been moved so that the openings 90 in the vent panel are moved out of alignment with the openings 92 in the top cover 86, with the vent panel 84 now blocking and closing the openings 92 in the top cover 86, thereby preventing escape of the waste heat out from the top of the sauna unit 10. In this closed position, the waste heat generated in the ventilation chamber 20 by the infrared heat sources 40 is directed downwardly through the infrared heat source fixtures, through the ceiling 36 and into the sauna enclosure 12, thereby increasing the temperature within the sauna enclosure 12. The user can adjust the amount of heat temperature level within the sauna enclosure 12 by moving the handle 82 of the vent assembly 80 between a fully open position through a range of adjusted positions and to the fully closed position. It should be noted that the vent panel 84 can be moved so that the openings 92 in the top cover 86 of the sauna unit 10 are only partially blocked or closed off, thereby allowing escape of some heat up through the top of the sauna unit 10.

As shown in FIGS. 8-12, the plurality of infrared heat sources 40 are provided in the ceiling 36 of the sauna enclosure 12. Additionally, one or more light sources 42, such as LED lights, are provided in the sauna enclosure 12 for lighting the interior of the sauna enclosure 12. A set of controls 44 on a control panel 43 is provided for operating the infrared heat sources 40 and light sources 42. As shown in FIG. 12, wires 46 are in connection with the set of controls 44 for delivering electric current to power the infrared heat sources 40 and light sources 42 along with the air intake fans 70, audio speaker, and light therapy device. One or more of the infrared heat sources 40 may be in connection with a timer 48, wherein the one or more infrared heat sources 40 are activated by the timer 48 for a period of time that may be either pre-programmed or selectively set by the user. The timer 48 may be set by operation of a timer control on the control panel and automatically deactivated after a predetermined amount of time has lapsed. All infrared heat sources 40 are automatically deactivated after the predetermined amount of time has lapsed. A power cord 100 with accompanying power plug 102 extends from the sauna unit 10 and is disposed for connection with a standard AC electric power outlet for powering the sauna unit 10. Referring specifically to FIG. 8, the infrared heat sources 40 and light sources 42 are covered by a protective screen 110, such as an aluminum mesh screen or stainless steel grill, that protects the user from inadvertently touching the infrared heat sources 40 and light sources 42 while still permitting passage of heat and light therethrough. The protective screen 110 is fastened to the ceiling within the sauna enclosure and may include hinges that attach to the screen 110 and the ceiling 36 to allow opening of the screen 110 and access to the infrared heat sources 40 and light sources 42 for bulb replacement and repairs. The interior of the sauna enclosure 12 includes a flat bottom surface 38 for the user to lie upon while receiving infrared therapy. A cushion and one or more pillows may be placed on the bottom surface 38 for additional comfort.

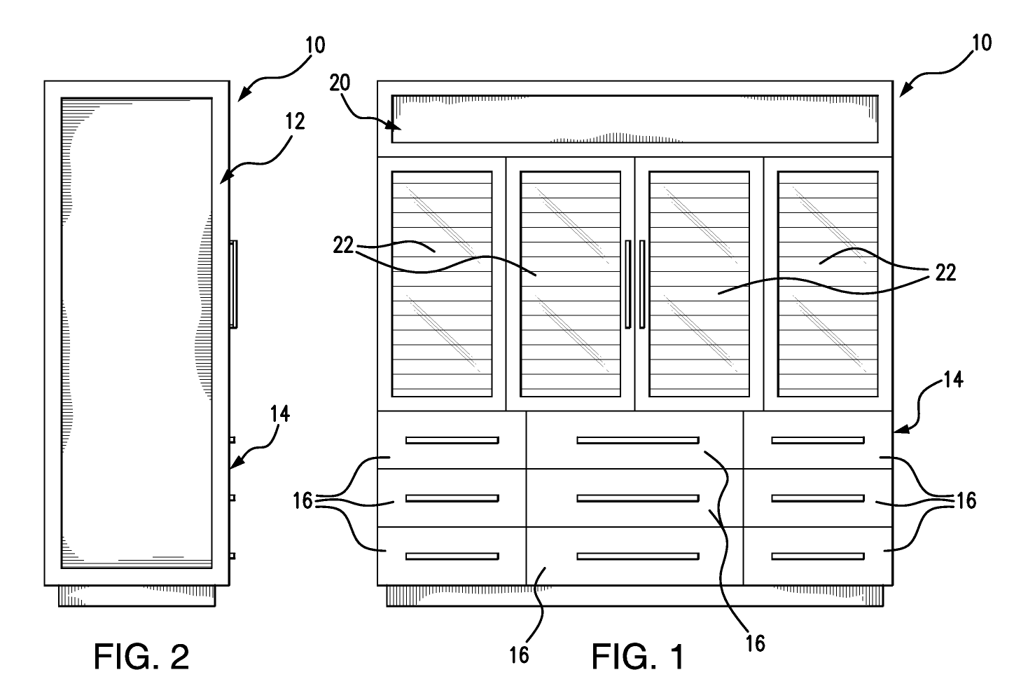

Referring to FIGS. 1 and 2, a first embodiment of the space saving sauna unit 10 is shown including a sauna enclosure 12 that is integrated with a piece of furniture 14, wherein the piece of furniture is a dresser with an arrangement of storage drawers 16 below the sauna enclosure 12. The sauna enclosure 12 is accessible through the front doors 16.

Referring to FIG. 3, another embodiment of the space saving sauna unit 10 is shown wherein the sauna unit is structured to have the appearance of an armoire. An open storage space 18 is provided directly below the sauna enclosure 12. Alternatively, this space can be filled with a cabinet.

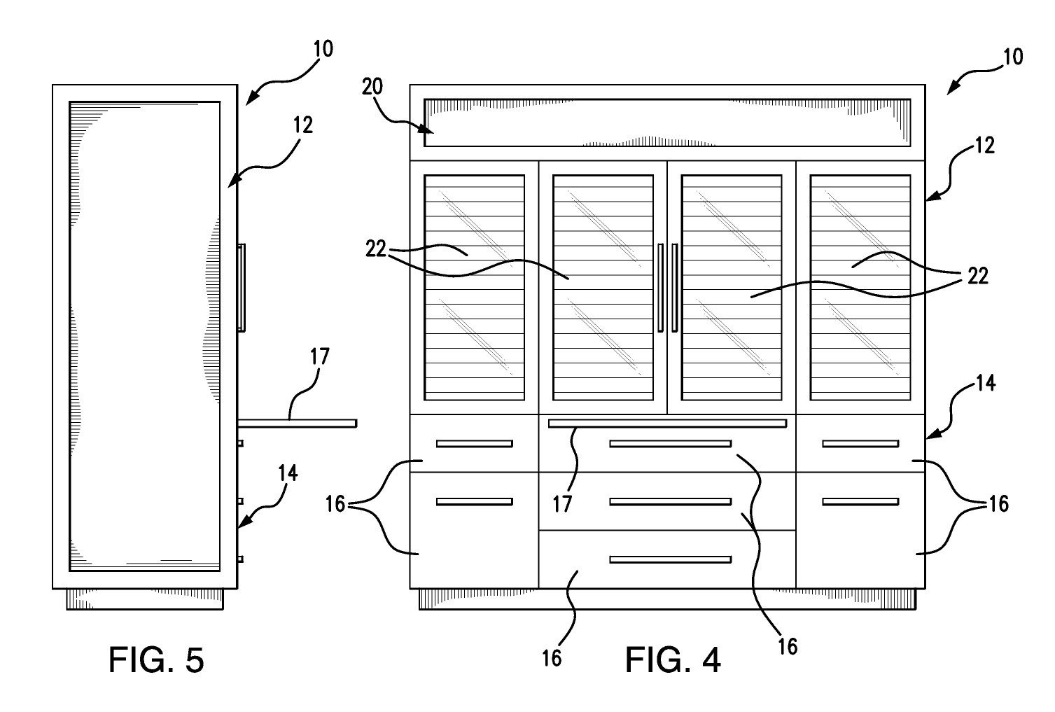

Referring to FIGS. 4 and 5, another embodiment of the space saving sauna unit 10 is shown including a sauna enclosure 12 is integrated with a desk. A pull-out desk top 17 is extendable to an operable position, as seen in FIG. 5 to allow a person to sit in a chair and use the pull-out desk top 17 as a desk surface. The desk top 17 can be pushed in and stored beneath the sauna enclosure 12 during non-use.

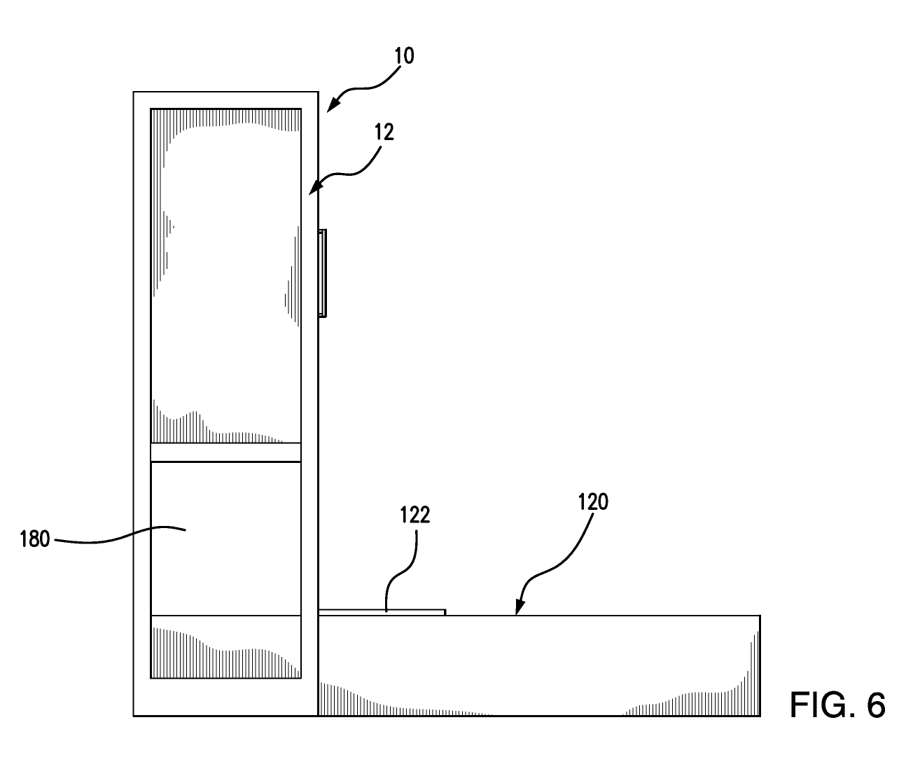

Referring to FIG. 6, another embodiment of the space saving sauna unit 10 is shown wherein the sauna enclosure 12 is integrated with a bed 120. A retractable or removable entry platform 122 is provided for stepping on by a user for facilitating easy entry into the sauna enclosure 12. In this embodiment, the storage space 180 shares the open space above the top (i.e., head) of the bed and beneath the sauna enclosure 12. Alternatively, a headboard (not pictured) may be installed at the head of the bed, below the sauna enclosure 12.

While the present invention has been shown and described in accordance with several preferred and practical embodiments, it is recognized that departures from the instant disclosure of the invention are fully contemplated within the spirit and scope of the invention which is not to be limited except as defined in the following claims as interpreted under the doctrine of equivalents.

What is claimed is:

1. A sauna apparatus comprising:
a sauna enclosure including a bottom surface, a ceiling and a plurality of walls extending from the bottom surface to the ceiling, an access opening sized and configured to allow ingress and egress of a user within the sauna enclosure, and at least one moveable door for selectively opening and closing the access opening;
at least one infrared heat source structured and disposed for generating heat within the sauna enclosure upon activation thereof;
a ventilation chamber adjacent to the sauna enclosure and communicating with the at least one infrared heat source for receiving waste heat generated by the at least one infrared heat source; and
at least one storage area below the sauna enclosure.

2. The sauna apparatus as recited in claim 1 further comprising:
a plurality of the infrared heat sources mounted within the ceiling of the sauna enclosure.

3. The sauna apparatus as recited in claim 2 wherein the ventilation chamber is directly above the sauna enclosure and the plurality of infrared heat sources are at least partially within the ventilation chamber.

4. The sauna apparatus as recited in claim 1 further comprising a vent assembly for selectively controlling release of the waste heat from the ventilation chamber to an exterior of the sauna apparatus and further for selectively controlling containment and direction of the waste heat into the sauna enclosure for increasing the temperature within the sauna enclosure.

5. The sauna apparatus as recited in claim 4 further comprising:
a manually operated control within the sauna enclosure for allowing a user to selectively operate the vent assembly while the user is within the sauna enclosure.

6. The sauna apparatus as recited in claim 1 further comprising:
an adjustably positionable light therapy device within the sauna enclosure for administering a light therapy skin treatment to a user while the user is within the sauna enclosure.

7. The sauna apparatus as recited in claim 1 further comprising:
at least one audio speaker within the sauna enclosure; and
at least one connector cable for connecting a digital audio storage and playback device for playing audio from the at least one speaker.

8. The sauna apparatus as recited in claim 1 wherein the sauna enclosure is integrated with an article of furniture.

9. The sauna apparatus as recited in claim 8 wherein the article of furniture is a dresser including a plurality of storage drawers below the sauna enclosure.

10. The sauna apparatus as recited in claim 8 wherein the article of furniture is a desk including a plurality of storage drawers and an extendable and retractable desk surface below the sauna enclosure.

11. The sauna apparatus as recited in claim 8 wherein the article of furniture is a bed.

12. The sauna apparatus as recited in claim 1 further including at least one light source within the sauna enclosure for lighting the interior of the sauna enclosure.

13. The sauna apparatus as recited in claim 1 further including a plurality of moveable doors for selectively opening and closing the access opening.

14. A sauna apparatus comprising:
a sauna enclosure including a bottom surface, a ceiling and a plurality of walls extending from the bottom surface to the ceiling, an access opening sized and configured to allow ingress and egress of a user within the sauna enclosure, and at least one moveable door for selectively opening and closing the access opening;
at least one infrared heat source structured and disposed for generating heat within the sauna enclosure upon activation thereof; and
at least one storage area below the sauna enclosure.

15. The sauna apparatus as recited in claim 14 further comprising:
a ventilation chamber adjacent to the sauna enclosure and communicating with the at least one infrared heat source for receiving waste heat generated by the at least one infrared heat source.

16. The sauna apparatus as recited in claim 15 further comprising:
a plurality of the infrared heat sources mounted within the ceiling of the sauna enclosure.

17. The sauna apparatus as recited in claim 16 wherein the ventilation chamber is directly above the sauna enclosure and the plurality of infrared heat sources are at least partially within the ventilation chamber.

18. The sauna apparatus as recited in claim 17 further comprising:
a vent assembly for selectively controlling release of the waste heat from the ventilation chamber to an exterior of the sauna apparatus and further for selectively controlling containment and direction of the waste heat into the sauna enclosure for increasing the temperature within the sauna enclosure.

19. The sauna apparatus as recited in claim 18 further comprising:
a manually operated control within the sauna enclosure for allowing a user to selectively operate the vent assembly while the user is within the sauna enclosure.
